(19) United States
(12) Patent Application Publication
(10) Pub. No.: US 2002/0102295 A1
(43) Pub. Date: Aug. 1, 2002

(54) Compositions for application to the skin or hair

(76) Inventors: Susan Niemiec, Yardley, PA (US); Snehal Shah, Cerritos, CA (US); Elvin R. Lukenbach, Flemington, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/939,885

(22) Filed: Aug. 27, 2001

Related U.S. Application Data
(63) Continuation-in-part of application No. 09/320,894, filed on May 27, 1999.

(51) Int. Cl. A61K 7/06; A61K 7/11; A61K 9/127

(52) U.S. Cl. 424/450; 424/70.11; 424/70.15; 424/70.13

(57) ABSTRACT

The present invention relates to a composition for application to the hair or skin which contains a conditioning polymer encapsulated in a lipid vesicle.

### CROSS REFERENCE TO RELATED APPLICATIONS

This application is a continuation-in-part of U.S. application Ser. No. 09/320,894 filed on May 27, 1999, which is incorporated by reference in its entirety.

### FIELD OF THE INVENTION

The invention relates to a composition for application to the hair or skin which contains a conditioning polymer encapsulated in a lipid vesicle.

### BACKGROUND OF THE INVENTION

Conditioning formulations are a popular means of cosmetic hair treatment. The intent of such formulations is to impart softness, gloss or shine, to reduce flyaway and to enhance cosmetic appeal of the hair such as adding volume. Examples of such conditioning agents include hydrolyzed proteins, quaternized cationic derivatives, cationic polymers, and silicones. These polymers leave a continuous smoothing film on the hair. By the virtue of their high spreading coefficients, silicones readily spread over the surface of hair forming a thin, uniform, hydrophobic film that increases luster and gloss. This film on the hair reduces the interfiber friction resulting in less need for combing force and, consequently, less damage during grooming. These conditioning agents, however, are usually washed away upon repeated washing with a cleaning shampoo and, thus, this cosmetic benefit is only temporary until the next application.

Similarly, polymers are used in providing benefits to the skin such as moisturization or emolliency. Dry skin may be made to feel smoother and softer by use of hydrophilic cationic polymers or hydrophobic emollients; a dry lubricious feeling is achieved by application of silicones; reduction of irritation and accelerated healing are achieved with proteins such as wheat protein and polysaccharide polymers such as chitosan and hyaluronic acid. Polymers are also used to cause other benefit agents to adhere to the skin, such as film forming polymers such as methyl cellulose

It is known in the art that the delivery of benefit agents may be deposited onto hair fibers by the use of specific lipid vesicles. See, e.g., U.S. Pat. Nos. 5,436,010 and 5,605,704. Applicants, however, have discovered that lipid vesicles can be used to deliver conditioning polymers to the hair and skin. Applicants have found that such conditioning polymers remain on the hair even after washing.

### SUMMARY OF THE INVENTION

In one aspect, the invention relates to a composition for application to the hair or skin which contains a conditioning polymer encapsulated in a lipid vesicle.

In another aspect, the invention features a method of conditioning, strengthening, or moisturizing the hair comprising topically applying a composition which contains a conditioning polymer encapsulated in a lipid vesicle.

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, percentages of ingredients are percentages by weight.

Conditioning Polymers

The conditioning polymers can be a natural, synthetic, or biosynthetic. What is meant by a “biosynthetic polymer” is a natural polymer that has been modified with one or more synthetic functional groups. What is meant by a “conditioning polymer” is a polymer that can effect one or more of the following attributes of hair: shine, volume, body, combability, style hold, and texture; or skin: moisturiziation, lubricity, increased healing, and texture. In one embodiment, the molecular weight of the conditioning polymer is from about 1000 to about 10 million such as from about 10,000 to about 1 millon.

Examples of natural polymers include, but are not limited to, polysaccharides and proteins. Proteins are made up of one or more polypeptide chains, which are in turn made up of many amino acid residues linked together by a peptide bonds having a molecular range from about 5000 to about 1 million (such as from about 1,000 to about 1 million). Such proteins may have both positively and negatively charged side chains, thus exhibiting amphoteric behavior. Also, quaternary ammonium groups can be covalently grafted on to the protein. Examples of proteins include, but are not limited to, those extracted from vegetable sources such as wheat and soy, and such proteins may be partially hydrolyzed to ease their use in formulations. Others proteins used include collagen, keratin, and silk.

Polysaccharides are made up of repeating monosaccharide units, or simple sugars, joined together in long linear or branched chains. Polysaccharides can be nonionic, anionic, or cationic. Major sources of polysaccharides include plant, animal and microbial. Examples of polysaccharides include, but are not limited to, starch from various sources such as corn, potato, tapioca, guar gum, agarose, mannan, dextran, carrageenan, alginie acid and xanthan. Other polysaccharides include chitin, carboxymethyl chitin, chitosan and polymers of hyaluronic acid a derivatives of hyaluronic acid.

Biosynthetic polymer derivatives of proteins include, but are not limited to, cationic and fatty acyl derivatives of natural polymers. Cellulose derived biosynthetic polymers include, but are not limited to, carboxymethyl cellulose, hydroxyethyl cellulose, hydroxypropyl cellulose, sodium carboxymethyl cellulose, methylcellulose, and methylhydroxyethylcellulose. Other examples include, but are not limited to, cationic hydroxyethyl cellulose, polyquaternium-10 (Polymer JR), cationic starch, polyquaternium-4 (Celquat), a copolymer of hydroxyethylcellulose and diallydimethyl ammonium chloride, Polyquaternium-24 (Quatrisoft Polymer LM-200), guar hydroxy-propyltrimonium-chloride, polyquaternium-29, crodocels, and siliconized wheat protein such as Crodasome W.

Synthetic polymers include, but are not limited to, homopolymers or copolymers of a variety of monomers such as vinyl acetate, methyl vinyl ether, acrylic acid and its esters, methacrylic acid and its esters, acrylamide, acrylonitrile, diallyldimethyl ammonium chloride, maleic acid, maleic anhydride, ethylene oxide, and vinyl pyrrolidone. Such polymers may be linear or branched.

Examples of acrylic polymers include, but are not limited to, Carbopol, polycarbophil, poly(acrylic acid), poly(methyl vinyl ether-co-methacrylic acid), poly acrylates, poly(2-hydroxyethyl methacrylate), poly(glyceryl mathacrylate), poly(methylmethacrylate), poly(methacrylate), poly(alkylcyanocrylate), poly(isohexylcyanoacrylate) and poly(isobutylcyanoacrylate), and polyquaternium-11 (Gafquat). Vinylpyrrolidone polymers include polyvinylpyrrolidone (PVP) which is a polymer that can be synthesized in various average molecular weights ranging from about 2,500 to about 1,200,000 (e.g., polyvinylpyrrolidone K-12, K15, K-17, K-25, K-30, K-90 which are available from International Specialties Products, Wayne, N.J.).

Another class of synthetic polymers include silicone polymers which include, but are not limited to, polydimethyl siloxanes such as dimethicones, dimethiconols, cyclomethicones, dimethicone copolyols which have a wide variety of molecular weights ranging from light liquids to gums, phenylmethicone, phenyl trimethicone, organosilicones, and simethicone. The silicone polymers may also comprise amino-functional groups known as amodimethicones or comprise quaternary groups such as quaternium-80.

In one embodiment, the silicone polymer is an organopolysiloxane elastomer. Organopolysiloxane elastomers are chain polymers having a degree of cross-linking sufficient to provide a rubber-like material. Suitable organopolysiloxane elastomers are disclosed in U.S. Pat. Nos. 5, 266,321 and 5,412,004, and the disclosure of which is incorporated herein by reference. The organopolysiloxanes have a three dimensional cross-linked structure and may have an average molecular weight in excess of 10,000 (e.g., between about 10,000 and 10,000,000).

Examples of organopolysilicone elastomers include crosslinked siloxane copolymers such as stearyl methyl-dimethyl siloxane copolymer (Gransil SR-CYC, available from Grant Industries, Elmwood Park, N.J.); Polysilicone-11 (i.e., a crosslinked silicone rubber formed by the reaction of vinyl terminated silicone and methylhydrodimethyl siloxane in the presence of cyclomethicone also available from Grant Industries), cetearyl dimethicone/vinyl dimethicone crosspolymer (i.e., a copolymer of cetearyl dimethicone crosslinked with vinyl dimethyl polysiloxane), dimethicone/phenyl vinyl dimethicone crosspolymer (i.e., copolymer of dimethylpolysiloxane crosslinked with phenyl vinyl dimethylsiloxane), and dimethicone/vinyl dimethicone crosspolymer (i.e., copolymer of dimethylpolysiloxane crosslinked with vinyl dimethylsiloxane).

In one embodiment, the composition contains from about 0.001 to about 20 percent (such as from about 0.1 to about 10 percent), by weight, of the conditioning polymer. In one embodiment, the lipid vesicle contains from about 0.1 to about 50 percent (such as from about 1 to about 25 percent), by weight, of the conditioning polymer.

Lipid Vesicles

As used herein, the term “lipid vesicle” means structures having one or more lipid bilayers that can encapsulate the conditioning polymer either within the core of the lipid vesicle or within the lipid bilayers. The terms “liposomes” and “lipid vesicles” are used interchangably herein. Examples of liposomes include, but are not limited to, unilamellar liposomes (having a single lipid bilayer surrounding a hydrophilic core), paucilamellar (having one or more bilayers surrounding a hydrophobic core), and multilamellar liposomes (having multiple bilayers surrounding a hydrophilic core). In one embodiment, the liposome does not contain any phospholipids (hereinafter a “non-phospholipid liposome”). In one embodiment, the liposome contains one or more dual chain lipids, optionally one or more single chain lipids, and optionally one or more sterols forming the lipid bilayer(s), and one or more hydrophilic liquids encapsulated within the lipid bilayer(s). The dual chain lipid(s) and single chain lipid(s) may either be cationic, amphoteric, or nonionic. Such ingredients are further defined below.

What is meant by a vesicle bilayers is the bilayer structure made of the lipid components of the vesicles (e.g., the single chain lipids, the dual chain lipids, and the sterols). Various combinations and ratios of the conditioning polymer(s), single chain lipid(s), dual chain lipid(s), sterol(s), hydrophilic liquid(s), and other optional additional agents may be used for preparing the lipid vesicles of the invention. The components of the lipid vesicle, and their respective weight ratio content therein may depend upon, for example, the final characteristics desired in the lipid vesicle, the properties of the different components in the system, the desired use(s) of the lipid vesicle, and/or the type of non-vesicle components (e.g., additional benefit agents) to be used with the lipid vesicle.

In one embodiment, the amounts are, based upon the total lipid vesicle, from about 40 percent to about 95 percent (such as from about 40 percent to about 60 percent) of the dual chain lipid(s); from about 1 percent to about 55 percent (such as from about 1 percent to about 35 percent) single chain lipid(s); from about 1 percent to about 50 percent (such as from about 1 percent to about 25 percent of sterol s); and from about 50 percent to about 99 percent (such as from about 60 percent to about 90 percent) hydrophilic liquids(s).

In one embodiment, the lipid vesicle is nonionic containing, based upon the total weight of the vesicle bilayers, from about 40 percent to about 60 percent (such as from about 40 percent to about 50 percent) of glyceryl distearate (a dual chain lipid); from about 10 percent to about 45 percent such as from about 10 percent to about 20 percent) of a polyoxyethylene-10-stearyl ether (a single chain lipid), and from about 5 percent to about 45 percent (such as from about 5 percent to about 25 percent) of cholesterol (a sterol).

In another embodiment, the lipid vesicle is a nonionic lipid vesicle contains, based upon the total weight of the vesicle bilayers, from about 45 to about 55 percent of glyceryl distearate, from about 1 percent to about 50 percent (such as from about 5 percent to about 25 percent) of cholesterol, and from about 18 percent to about 28 percent of polyoxyethylene-10-stearyl ether.

In one embodiment, the lipid vesicle is a cationic lipid vesicle containing, based upon the total weight of the vesicle bilayer, from about 25 percent to about 95 percent (such as from about 30 percent to about 65 percent) glyceryl distearate; from about 1 percent to about 45 percent (such as from about 5 percent to about 35 percent) of a polyoxyethylene-10-stearyl ether; from about 1 percent to about 40 percent (such as from about 5 percent to about 25 percent) of a cholesterol, and from about 1 percent to about 45 percent (such as from about 2 percent to about 25 percent) of a di(soyoylethyl) hydroxyethylmonium methosulfate (DSHM, a cationic dual chain lipid).

In another embodiment the lipid vesicle is a cationic lipid vesicle containing, based upon the total weight of the vesicle bilayers, from about 25 percent to about 60 percent (such as from about 23 percent to about 27 percent) of a nonionic dual chain lipid such as glyceryl dilaurate (“GDL”); from about 5 percent to about 45 percent (such as from about 23 percent to about 27 percent) of another nonlonic dual chain lipid such as glyceryl distearate (“GDS”); from about 1 percent to about 40 percent (such as from about 13 percent to about 17 percent) of a sterol such as cholesterol, from about 5 percent to about 40 percent (such as from about 20 percent to about 25 percent) of a nonionic single chain lipid such as polyoxyethylene-10-stearyl ether, and from about 1 percent to about 45 percent (such as from about 10 percent to about 15 percent) of a cationic dual chain lipid such as di(soyoylethyl)hydroxyethylmonium methosulfate.

Lipid vesicles may preferably be prepared by mixing appropriate amounts of the single chain lipids, the dual chain lipids, and sterols under conditions sufficient to produce a homogeneous mixture. While the temperature for mixing may depend upon, for example, the melting points of the predominate lipids, typically the lipid vesicle may be prepared under temperatures of from about 65° C. to about 80° C. and under ambient pressure conditions. In order to produce a lipid vesicle having improved consistency, it is more preferable to mix the single chain lipids, the dual chain lipids, and the sterols under high shear in, for example, an apparatus as described in U.S. Pat. Nos. 5,013,497, 3,176,964, 3,408,050, and 3,926,413 which is incorporated by reference herein.

Lipid vesicles containing both non-ionic and cationic lipids may be prepared by first preparing the nonionic lipid vesicle as described above, followed by mixing the cationic lipids therewith at the phase transition temperature of the combined lipids in a mixer, such as a Caframo mixer. In a preferred alternative embodiment, the dual chain lipids, the single chain lipids, the sterols and the cationic lipids may be mixed simultaneously.

Dual Chain Lipid

In one embodiment, the lipid vesicle comprises one or more dual chain lipids, which may be comprised of a polar head group and two nonionic hydrophobic chains, two cationic hydrophobic chains, or one nonionic and one cationic hydrophobic chain.

Examples of suitable nonionic dual chain lipids include, but are not limited to, glyceryl diesters, alkoxylated amides, and mixtures thereof.

Examples of suitable glyceryl diesters include those glyceryl diesters having from about 10 carbon atoms to about 30 carbon atoms (such as from about 12 carbon atoms to about 20 carbon atoms) Preferred glyceryl diesters include, but are not limited to, glyceryl dilaurate (“GDL”), glyceryl dioleate, glyceryl dimyristate, glyceryl distearate (“GDS”), glyceryl sesquioleate, glyceryl stearate lactate, and mixtures thereof.

Examples of suitable alkoxylated amides include, but are not limited to, those which conform to the structure shown below in Formula I:

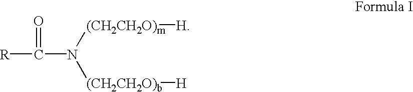

wherein R is a unbranched alkyl group having from about 8 carbon atoms to about 30 carbon atom (such as from about 12 carbon atoms to about 24 carbon atoms), m is an integer of from about 0 to about 100, and b is an integer of from about 0 to about 100, with the proviso that the sum of m and b is from about 8 to about 100. An exemplary member of this class is PEG-6 Cocoamide (wherein RCO represents the fatty acids derived from coconut oil and both m and b, respectively, have an average value of about 6).

Examples of suitable cationic dual chain lipids include, but are not limited to, those bilayer-forming cationic lipids that contain two unsaturated fatty acid chains having from about 8 to about 26 carbon atoms.

Examples of cationic dual chain lipids include, but are not limited to, di(soyoylethyl) hydroxyethylmonium methosulfate (DSHM); N-[I-(2,3-dioleyloxy)propyl]-N,N,N-trimethylammonium bromide (DOTMA); 1,2-dimyristyloxypropyl-N,N-dimethyl-hydroxyethyl ammonium bromide (DMRIE); [N-(N,N′-dimethylaminoethane)carbamoyl] cholesterol (DC-Chol); dioctadecylamidoglycyl spermidine (DOGS); dimethyl dioctadecylammonium bromide (DDAB); dioleoyl phosphatidylethanolamine (DOPE); 2,3-dioleoyloxyl-N[2(sperminecarbozamide-O-ethyl]-N,N-dimethyl-propanaminium trifluoroacetate (DOSPA); I-[2-(oleoyloxy)-ethyl]-2-oleyl-3-(2-hydroxyethyl)imidazolinium chloride (DOTIM); 1,2-dioleoyloxy-3-(trimethylammonio)propane (DOTAP); 1,2-diacyl-3-trimethylammonium propane (TAP); 1,2-diacyl-3-dimethylammonium propane (DAP); quaternary dimethyldiacyl amines wherein the acyl groups have from about 8 carbon atoms to about 30 carbon atoms (such as from about 10 carbon atoms to about 24 carbon atoms), derivatives thereof such as ammonium derivatives such as dicocodimonium chloride (Quaternium 34), dimethyl dihydrogenated tallow ammonium chloride (Quaternium 18), and decyl dimethyl octyl ammonium chloride (Quaternium 24), and mixtures thereof.

Several of these cationic dual chain lipids, such as TAP and DAP, may possess a variety of types of chain groups having carbon atom to number of saturated bonds ratios of, for example, 14:0, 16:0, 18:0, and 18:1, as well as a variety of types of acyl groups having from about 10 carbon atoms to about 18 carbon atoms such as dimyristoyl, dipalmitoyl, distearoyl, and dioleoyl.

In one embodiment, the amount of dual chain lipids in the vesicle bilayer may range from, based upon the total weight of the vesicle bilayer, from about 0 percent to about 95 percent (such as from about 10 percent to about 65 percent).

Single Chain Lipid

In one embodiment, the lipid vesicle comprises one or more single chain lipid(s), which may be comprised of a polar head group and a non-ionic hydrophilic chain or a cationic hydrophilic chain.

Examples of suitable nonionic single chain lipids include, but are not limited to glyceryl monoesters;

polyoxyethylene fatty ethers wherein the polyoxyethylene head group has from about 2 to about 100 oxyethylene groups and the fatty acid tail group has from about 10 to about 26 carbon atoms; alkoxylated alcohols wherein the alkoxy group has from about 1 carbon atoms to about 200 carbon atoms and the fatty alkyl group has from about 8 carbon atom to about 30 carbon atoms (such as from about 10 carbon atoms to about 24 carbon atoms); alkoxylated alkyl phenols wherein the alkoxy group has from about 1 carbon atoms to about 200 carbon atoms and the fatty alkyl group has from about 8 carbon atom to about 30 carbon atoms (such as from about 10 carbon atoms to about 24 carbon atoms); polyoxyethylene derivatives of polyol esters; alkoxylated acids wherein the alkoxy group has from about 1 carbon atoms to about 200 carbon atoms and the fatty acyl group has from about 8 carbon atom to about 30 carbon atoms (such as from about 10 carbon atoms to about 24 carbon atoms); and 10 mixtures thereof.

Examples of suitable glyceryl monoester nonionic single chain lipids preferably include, but are not limited to, those glyceryl monoesters having from about 10 carbon atoms to about 30 carbon atoms (such as from about 12 carbon atoms to about 20 carbon atoms), and mixtures thereof. Preferred glyceryl monoesters include glyceryl caprate, glyceryl caprylate, glyceryl cocoate, glyceryl erucate, glyceryl hydroxystearate, glyceryl isostearate, glyceryl lanolate, glyceryl laurate, glyceryl linolate, glyceryl myristate, glyceryl oleate, glyceryl PABA, glyceryl palmitate, glyceryl ricinoleate, glyceryl stearate, and mixtures thereof.

Examples of suitable polyoxyethylene fatty ether nonionic single chain lipids include, but are not limited to, polyoxyethylene cetyl ether, polyoxyethylene stearyl ether, polyoxyethylene cholesterol ether, polyoxyethylene lauryl ether, and mixtures thereof. Preferred polyoxyethylene fatty ethers include polyoxyethylene stearyl ether, polyoxyethylene myristyl ether. In one embodiment, each ether has from about 3 to about 10 oxyethylene units.

Suitable examples of an alkoxylated alcohol nonionic single chain lipid include, but are not limited to, those that are useful as nonionic surfactants and have the structure shown in formula II below:

R₅—(OCH₂CH₂)y—OH Formula II

wherein R₅ is an unbranched alkyl group having from about 10 to about 24 carbon atoms and y is an integer between about 4 and about 100 (such as from about 10 to about 100). A preferred alkoxylated alcohol is the species wherein R₅ is a lauryl group and y has an average value of 23 (Laureth 23, available from Uniqema, Inc. of Wilmington, Del. under the tradename BRIJ 35).

Suitable examples of an alkoxylated alkyl phenols nonionic single chain lipid include, but are not limited to, those which generally conform to the structure shown in Formula III below:

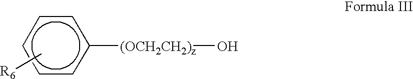

wherein R₆ is an unbranched alkyl group having from about 10 to about 24 carbon atoms and z is an integer of from about 7 and 120 (such as from about 10 to about 100). A preferred member of this class is the species wherein R₆ is a nonyl group and z has an average value of about 14 (Nonoxynol-14, available under the tradename MAKON 14 from the Stepan Company of Northfield, Ill.).

Suitable polyoxyethylene derivatives of polyol ester single chain nonionic lipids include, but are not limited to, those wherein the polyoxyethylene derivative of polyol ester that (1) is derived from (a) a fatty acid containing from about 8 to about 22 (such as from about 10 to about 14 carbon atoms), and (b) a polyol selected from sorbitol, sorbitan, glucose, α-methyl glucoside, polyglucose having an average of about 1 to about 3 glucose residues per molecule, glycerine, pentaerythritol and mixtures thereof, (2) contains an average of from about 10 to about 120 (such as from about 20 to about 80 oxyethylene units); and (3) has an average of about 1 to about 3 fatty acid residues per mole of polyoxyethylene derivative of polyol ester.

Examples of preferred polyoxyethylene derivatives of polyol) esters include, but are not limited to PEG-80 sorbitan laurate and Polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the tradename, “Atlas G-4280.” Polysorbate 20, which is the laurate monoester of a mixture of sorbitol and sorbitol anhydrides condensed with approximately 20 moles of ethylene oxide, is available commercially from ICI Surfactants of Wilmington, Del. under the tradename “Tween 20.” Another exemplary polyol ester is sorbitan stearate, which is available from Uniqema, Inc. under the tradename SPAN 60.

Suitable examples of alkoxylated acid single chain, nonionic lipids include, but are not limited to, the esters of an acid, most usually a fatty acid, with a polyalkylene glycol. Exemplary materials of this class are, polyoxyethylene laurate such as PEG-8 laurate, polyoxyethylene dilaurate, polyoxyethylene stearate, polyoxyethylene distearate.

Preferred single chain nonionic lipids include polyoxyethylene fatty ethers such as polyoxyethylene stearyl ether, polyoxyethylene myristyl ether, and polyoxyethylene lauryl ether whereby each ether has from about 5 to about 10 oxyethylene units and glyceryl monoesters such as glyceryl laurate, glyceryl myristate, and glyceryl stearate, and mixtures thereof.

Examples of suitable cationic single chain lipids nonexclusively include, but are not limited to, quaternary trimethylmonoalkyl amines, wherein the alkyl group has from about 8 carbon atoms to about 30 carbon atoms (such as from about 10 carbon atoms to about 24 carbon atoms), and derivatives and mixtures thereof such as ammonium derivatives such as stearamidopropyl dimethyl (myristyl acetate) ammonium chloride (Quaternium 70), triethyl hydrogenated tallow ammonium chloride (Quaternium 16), benzalkonium chloride, and derivatives and mixtures thereof.

In one embodiment, the amount of single chain lipids in the vesicle bilayer may range from, based upon the total weight of the vesicle bilayer, from about 0 percent to about 70 percent (such as from about 1 percent to about 30 percent).

Sterols

In one embodiment, the lipid vesicle contains one or more sterols. Examples of suitable sterols include, but are not limited to, cholesterol and salts and esters thereof, phytocholesterol, hydrocortisone, alpha-tocopherol, betasitosterol, bisabolol and mixtures thereof.

In one embodiment, the amount of sterol in the vesicle bilayer may range from, based upon the total weight of the vesicle bilayer, from about 0 percent to about 50 percent (such as from about 1 percent to about 15 percent).

Hydrophilic Liquids

In one embodiment, the lipid vesicle comprises one or more hydrophilic liquids such as water, polar solvents, and mixtures thereof. Examples of polar solvents include, but are not limited to, glycols such as glycerin, alcohols such as those having from about 2 carbon atoms to about 6 carbon atoms (such as ethanol, propanol, and isoproanol), propylene glycol, sorbitol, oxyalkylene polymers such as PEG 4, and mixtures thereof.

In one embodiment, the amount of hydrophilic liquids in the lipid vesicle may range from, based upon the total weight of the vesicle bilayer, from about 1 percent to about 99 percent (such as from about 40 percent to about 90 percent).

Compositions for Application to the Hair and/or Skin

The above lipid vesicle are contained within compositions for application to the hair and/or skin. In one embodiment, the lipid vesicle is present in an amount effective to enable a sufficient amount of the conditioning polymer into and/or onto the hair or skin.

The amount of the lipid vesicle contained within the composition will vary with the type and amount of conditioning polymer, the intended usage of the final composition (e.g., therapeutic or maintenance regimen), the amount of detergent present, and the sensitivity of the individual user to the composition. Typically, the composition will contain from about 0.001% to about 99%, by weight (such as about 0.1% to about 25% or from about 1% to about 10%) of lipid vesicles.

The composition of this invention can be formulated in a variety of dosage forms for topical application to the hair and/or skin that include, but are not limited to, shampoos, body or facial wash, leave-on conditioner compositions, and rinse-off conditioner compositions. The compositions can be toners, lotions, creams, ointments, solutions, and pastes.

While the frequency and amount of the lipid vesicle to be applied will depend upon, for example, the type and amount of conditioning polymer, the intended usage of the final composition, i.e. therapeutic versus maintenance regimen, the amount and type of detergent present, and the sensitivity of the individual user to the composition, typically the composition of the present invention should be topically applied to hair or skin at regular intervals such as from about 1 to about 14 times per week.

In one embodiment wherein the composition is a shampoo, the shampoo is applied to wet hair, and the hair is washed in accordance with known practices. In a further embodiment, the composition remains on the hair for greater than about 0 to about 10 minutes such as from about 30 seconds to about 5 minutes before rinsing.

Further within the scope of the invention are kits that are comprised of the lipid vesicle, an optional benefit agent, and an optional detergent as well as instructions for their use. In one embodiment, the kit may be comprised of some or all of the materials for forming the lipid vesicle packaged separately or in pre-mixed combinations as well as instructions explaining the preparation of the delivery system. In yet other embodiments, such kits can further comprise a benefit agent and/or a detergent, wherein the benefit agent is either premixed, i.e. with the lipid vesicle components or the detergent, or provided in a separate container therefrom.

Detergents

In one embodiment, the composition further comprises a detergent. What is meant by a “detergent” is a surfactant and/or soap. Examples of surfactants include, but not limited to, anionic surfactants, nonionic surfactants, cationic surfactants, amphoteric surfactants (including betaine surfactants and zwitterionic surfactants) and mixtures thereof.

Examples of suitable anionic surfactants include, but are not limited to, compounds in classes known as alkyl sulfates, alkyl ether sulfates, sulfate esters of an alkylphenoxy polyoxyethylene ethanol, alpha-olefin sulfonates, betaalkyloxy alkane sulfonates, alkyl arylsulfonates, alkyl carbonates, alkyl ether carboxylates, fatty acids, alkyl sulfosuccinates, alkyl ether sulfosuccinates, alkyl sarcosinates, alkyl phosphates, alkyl ether phosphates, octoxynol phosphates, nonoxynol phosphates, alkyl taurates, fatty methyl taurides, sulfated monoglycerides, fatty acid amido polyoxyethylene sulfates, acyl amino acids, and acyl isethionates and mixtures thereof. In one embodiment, the anionic surfactant is present in the composition as a neutralized salt such as sodium salts, potassium salts, ammonium salts, lithium salts, alkyl ammonium salts, or hydroxyalkyl ammonium salts.

Preferred anionic surfactants are alkyl sulfates, alkyl ether sulfates, alkyl phosphates, acyl amino acid salts such as N-acyl-L-glutamate, α-olefin sulfonates, alkyl sarcosinates, alkyl benzene sulfonates, acyl isethionates, alkyl sulfosuccinates, acyl methyl taurides, and mixtures thereof.

Examples of suitable nonionic surfactants include, but are not limited to, polysorbate 20, long chain alkyl glucosides having alkyl groups containing about 8 carbon atoms to about 22 carbon atoms, coconut fatty acid monoethanolamides such as cocamide MEA, coconut fatty acid diethanolamides, and mixtures thereof.

Examples of suitable cationic surfactants include, but are not limited to, quaternary ammonium surfactants and quaternary amine surfactants that are not only positively charged at the pH of the composition, which generally is about pH 10 or lower, but also are soluble in the composition. Preferred cationic surfactants include, but are not limited to, the n-acylamidopropyl dimethylamine oxides such as cocamidopropylamine oxide.

Examples of suitable amphoteric surfactants include, alkyl amphocarboxylates, alkyl betaines, amidoalkylbetaines, amidoalkylsultaines, alkyl amphophosphates, alkyl phosphobetaines, amido-alkyl phosposphobetaines, alkyl pyrophosphobetaines, amido-alkyl pyrophosposphobetaines, carboxyalkyl alkyl polyamines, and mixtures thereof. Preferred amphoteric surfactants include amidoalkylbetaines such as cocamidopropyl betaine available commercially from Goldschmidt Chemical Corporation of Hopewell, Va. under the tradename “Tegobetaine L-7”; alkyl amphocarboxylates having from about 8 carbon atoms to about 18 carbon atoms in the alkyl group such as Sodium Cocoamphopropionate available commercially from Mona industries Inc. of Paterson, N.J. under the tradename “Monateric CA-35”.

Examples of suitable soaps include, but are not limited to, fatty acids reacted with potassium, sodium, ammonium, lithium, triethanol amine bases to form soaps such as sodium cocoate or triethanolamine cocoate.

In a preferred embodiment, the detergent is comprised of a mixture of, based upon the total weight of the detergent, from about 0.1 percent to about 20 percent (such as from about 5 percent to about 15 percent) anionic surfactants, from about 0 percent to about 10 percent (such as from about 1 percent to about 7 percent) nonionic surfactants, from about 0 percent to about 5 percent (such as from about 0 percent to about 4 percent) cationic surfactants, and from about 0.1 percent to about 15 percent (such as from about 1 percent to about 10 percent) amphoteric surfactants.

Benefit Agents

In one embodiment, the compositions of the present invention further comprises an benefit agent in addition to the conditioning polymer. The additional benefit agent may be contained either internal or external to the lipid vesicle.

In one embodiment, the benefit agent is a hair conditioner, a hair softener, or a hair mositurizer. Examples of suitable hair conditioners include, but are not limited to, quaternized compounds such as behenamidopropyl PG-dimonium chloride, tricetylammonium chloride, dihydrogenated tallowamidoethyl hydroxyethylmonium methosulfate, and mixtures thereof as well as lipophilic compounds like cetyl alcohol, stearyl alcohol, hydrogenated polydecene, and mixtures thereof. Examples of suitable hair softeners include, but are not limited to, silicone compounds such as those that are either non-volatile or volatile and those that are water soluble or water insoluble. Examples of suitable hair moisturizer include, but are not limited to, panthenol, panthenyl ethyl ether, pythantriol, and mixtures thereof.

In one embodiment, the benefit agent includes those benefit agents that are effective in the treatment of dandruff, seborrheic dermatitis, and psoriasis. Examples of such suitable benefit agents include, but are not limited to, zinc pyrithione, selenium sulfide, sulfur, salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazole nitrite and any possible stereo isomers and derivatives thereof, anthralin, piroctone olamine (octopirox), selenium sulfide, ciclopirox olamine, anti-psoriasis agents such as vitamin D analogs such as calcipotriol, calcitriol, and tacaleitrol, retinoids such as retinol, retinyl palmitate, retinyl acetate, and retinoic acid; corticosteroids such as hydrocortisone, clobetasone butyrate, clobetasol propionate, and derivative and mixtures thereof.

In one embodiment, the benefit agent includes those benefit agents that are effective in the treatment treating hair loss, such as hair loss resulting from alopecia. As used herein, “hair loss treatment agents” shall include agents capable of growing hair and/or agents capable of preventing the loss of hair.

Examples of hair loss treatment agents include, but are not limited to, potassium channel openers or peripheral vasodilators such as minoxidil, diazoxide, and N*-cyano-N-(tert-pentyl)-N′-3-pyridinyl-guanidine (P-1075); vitamins such as vitamin E and vitamin C and derivatives thereof such as vitamin E acetate and vitamin C palmitate; hormones such as erythropoietins and prostaglandins (e.g., prostaglandin El and prostaglandin F2-alpha); fatty acids such as oleic acid; diruretics such as spironolactone; heat shock proteins such as HSP 27 and HSP 72; calcium channel blockers such as verapamil HCL, nifedipine, and diltiazemamiloride; immunosuppressant drugs such as cyclosporin and Fk-506; 5 alpha-reductase inhibitors such as finasteride; growth factors such as EGF, IGF and FGF; transforming growth factor beta; tumor necrosis factor; non-steroidal anti-inflammatory agents such as benoxaprofen; retinoids such as tretinoin; cytokines such as IL-6, IL-1 alpha, and IL-1 beta; cell adhesion molecules such as ICAM; glucorcorticoids such as betametasone; botanical extracts such as aloe, clove, ginseng, rehmannia, swertia, sweet orange, zanthoxylum, Serenoa repens (saw palmetto), Hypoxis rooperi, stinging nettle, pumpkin seeds, and rye pollen; other botanical extracts such as sandlewood, red beet root, chrysanthemum, rosemary, and burdock root; homeopathic agents such as Kalium Phosphoricum D2, Azadirachta indica D2, and Joborandi DI; genes for cytokines, growth factors, and male-pattern baldness; antifungals such as ketoconazole and elubiol; antibiotics such as streptomycin; proteins inhibitors such as cycloheximide; acetazolamide; benoxaprofen; cortisone; diltiazem; hexachlorobenzene; hydantoin; nifedipine; penicillamine; phenothiazines; pinacidil; psoralens; verapamil; zidovudine; alpha-glucosylated rutin having at least one of the following rutins: quercetin, isoquercitrin, hespeddin, naringin, and methylhesperidin; and flavonoids and transglycosilated derivatives thereof; and mixtures thereof. Preferred hair loss treatment agents include minoxidil, finasteride, retinoids, ketoconazole, elubiol or mixtures thereof.

In one embodiment, the benefit agent includes those benefit agents that are effective in inhibiting hair growth. Examples of benefit agents suitable for use in inhibiting hair growth include: serine proteases such as trypsin; vitamins such as alpha-tocophenol (vitamin E) and derivatives thereof such as tocophenol acetate and tocophenol palmitate; antineoplastic agents such as doxorubicin, cyclophosphamide, chlormethine, methotrexate, fluorouracil, vincristine, daunorubicin, bleomycin and hydroxycarbamide; anticoagulants such as heparin, heparinoids, coumaerins, detran and indandiones; antithyroid drugs such as iodine, thiouracils and carbimazole; lithium and lithium carbonate; interferons such as interferon alpha, interferon alpha-2a, and interferon alpha-2b; retinoids such as retinol and isotretinoin; glucocorticoids such as betamethasone and dexamethosone; antihyperlipidaemic drugs such as triparanol and clofibrate; thallium; mercury; albendazole; allopurinol; amiodarone; amphetamines; androgens; bromocriptine; butyrophenones; carbamazepine; cholestyramine; cimetidine; clofibrate; danazol; desipramine; dixyrazine; ethambutol; etionamide; fluoxetine; gentamicin, gold salts; hydantoins; ibuprofen; impramine; immunoglobulins; indandiones; indomethacin; intraconazole; levadopa; maprotiline; methysergide; metoprolol; metyrapone; nadolol; nicotinic acid; potassium thiocyanate; propranolol; pyridostimine; salicylates; sulfasalazine; terfeinadine; thiamphenicol; thiouracils; trimethadione; troparanol; valproic acid; and mixtures thereof. Preferred hair growth inhibitory agents include serine proteases, retinol, isotretinoin, betamethoisone, alpha-tocophenol, and derivatives or mixtures thereof.

In one embodiment, the benefit agent includes those benefit agents that are effective in treating acne and for reducing the signs of aging such as wrinkles, fine lines, and other manifestations of photodamage and aging.

Examples of suitable anti-aging agents include, but are not limited to inorganic sunscreens such as titanium dioxide and zinc oxide; organic sunscreens such as octyl-methyl cinnamates and derivatives thereof; retinoids; vitamins such as vitamin E, vitamin A, vitamin C, vitamin B, and derivatives thereof such as vitamin E acetate, vitamin C palmitate, and the like; antioxidants including beta carotene, alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono 1,4-lactone, gluconic acid, gluconolactone, glucuronic acid, glucurronolactone, glycolic acid, isopropyl pyruvate, methyl pyruvate, mucic acid, pyruvic acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, nice, safflower, and mixtures thereof.

Examples of anti-aging agents include, but are not limited to, retinoids, anti-oxidants, alpha-hydroxy acids and beta-hydroxy acid.

The amount of benefit agent to be combined with the lipid vesicle may vary depending upon, for example, the resulting benefit desired and the sensitivity of the user to the benefit agent. However, typically, the composition contains, based upon the total weight of the composition, from about 0.001 percent to about 20 percent (such as from 0.01 percent to about 5 percent) of the benefit agent. If the benefit agent is contained within the lipid vesicle, the lipid vesicle contains from about 0.06 percent to about 60 percent (such as from about 0.6 percent to about 30 percent) by weight of the benefit agent based upon the total weight of the lipid vescicle.

Suspending Agents

The suspending agent is preferably used in an amount effective for suspending the lipid vesicles and/or external benefit agents within the composition. Although the amount of suspending agent may vary dependent upon type of lipid vesicle and/or benefit agent, viscosity of the formulation desired, and the stability of the formulation, typically the amount of suspending agent may range, based upon the total weight of the composition, from about 0.001 percent to about 10 percent (such as from about 0.1 percent to about 1 percent).

Examples of suitable suspending agents include, but are not limited to:

1) acrylate polymers and copolymers thereof such as the Acrylates/Aminoacrylates C10-30 Alkyl PEG-20 Itaconate copolymer available commercially from National Starch and Chemical Corporation of Bridgewater, N.J. under the trade name “Structure Plus”;

2) fatty acyl derivatives, wherein the acyl group is of the Formula IV:

R₁₀CO— Formula IV

wherein R₁₀ comprises a carbon chain having from about 7 to about 21 carbon atoms that is either saturated or unsaturated and is either substituted or unsubstituted with, for example, hydroxyl groups; Examples of suitable fatty acyl derivatives include ethylene glycol distearate, ethylene glycol monostearate, and alkanolamides such as cocamide MEA.

3) esters of long chain fatty acids, wherein the fatty acids is of the Formula V:

R₁₁COOR₁₂ Formula V

wherein R₁₁ is an alkyl group having from 8 carbon atoms to about 30 carbon atoms and R12 is an alkyl group having from 8 carbon atoms to about 30 carbon atoms, such as stearyl stearate;

4) alkyl dimethylamine oxides wherein the alkyl group has from about 8 carbon atoms to about 18 carbon atoms;

5) methylvinylether/maleic anhydride copolymer crosslinked with 1,9-decadiene PolyVM/MA (PVM/MA decadiene crosspolymer) available from International Specialty Products under the tradename, “Stabileze 06 & OM;”

6) cellulose derivatives such as methylcellulose, hydroxybutyl methylcellulose, hydroxypropylcellulose, hydroxypropyl methylcellulose, hydroxyethyl ethylcellulose, hydroxyethyl cellulose, and mixtures thereof;

7) Distearyl Phthalic Amide available from Stepan Company under the tradename “Stepan SAB-2,” and Di(hydrogenated) Tallow Phthalic Amide available from the same under the tradename “Stepan TAB-2”;

8) primary amines having a fatty alkyl group with at least 16 carbon atoms such as palmitate amine and stearamine;

9) polyacrylic acids such as carbomers, which are available from B. F. Goodrich Company under the tradename, “Carbopol”;

10) polysaccharide gums such as xanthan gum;

11) colloidal clays such as benzyl dimethyl hydrogenated tallow ammonium montmorillonite (Bentone 27);

12) colloidal silica; and

13) mixtures thereof.

Preferred suspending agents include carbomer, hydroxyethyl cellulose, methylvinylether/maleic anhydride copolymer crosslinked with 1,9-decadiene PolyVM/MA (PVM/MA decadiene crosspolymer), and Acrylates/Aminoacrylates C10-30 Alkyl PEG-20 Itaconate Copolymer.

The following is a description of the manufacture of and testing of lipid vesicles and compositions of the present invention. other compositions of the invention can be prepared in an analogous manner by a person of ordinary skill in the art.

### EXAMPLE 1

Preparation of Lipid Vesicles Containing Hydrophilic and/or Hydrophobic Polymers

Table 1 describes lipid vesicles used in the following examples.

TABLE 1
Lipid polymeric vesicles

| | | | | |
|---|---|---|---|---|
| Component | F#1 | F#2 | F#3 | Ranges % |

Lipid Phase

| | | | | |
|---|---|---|---|---|
| Glyceryl | 45 | 54.6 | 22.5 | 0-90 |
| distearate | | | | |
| Cholesterol | 15 | 18.2 | 7.5 | 0-60 |
| Polyoxyethylene- | 20 | 24.2 | 10 | 0-80 |
| 10-stearyl ether | | | | |
| DSHM* | 20 | 3.0 | 10 | 0-60 |
| Phenyl | 0 | 0 | 50 | 0-80 |
| Trimethicone | | | | |
| Sub Total | 100 | 100 | 100 | 100 |

Aqueous Phase

| | | | | |
|---|---|---|---|---|
| Di water | 68.1 | 68.1 | 99.6 | 10-99.99 |
| Methyl Paraben | 0.2 | 0.2 | 0.05 | 0-2 |
| Propyl Paraben | 0.2 | 0.2 | 0.05 | 0-2 |
| PVP K-30 | 31.5 | 31.5 | 0 | 0-80 |
| Sub Total | 100 | 100 | 100 | 100 |

*Dual Chain Cationic lipid: Di(soyoylethyl) hydroxyethylmonium methosulfate

Each of the above formulations was made by mixing appropriate amounts of the components of the lipid phase in a beaker at 65° C. until the lipids melted. The resulting melt was then drawn into a syringe, which was preheated in a water-bath to 65° C. In a second beaker the aqueous phase was combined and mixed until uniform. A second syringe the aqueous phase was preheated in a water-bath to 60° C. The two syringes were then connected via a 3-way metal stopcock. The ratio of aqueous phase to lipid phase was about 80:20 or 8 ml of aqueous phase to 2 ml of lipid phase. The ratio can be modified to range from about 90:10 to about 50:50. After injecting the aqueous phase components into the lipid phase syringe, the resulting mixture was rapidly mixed back and forth between the two syringes several times until the contents cooled to about 25-30° C.

### EXAMPLE 2

Preparation of Lipid Vesicles Containing Dual Chain and Single Chain Lipid Components and Hydrophilic and/or Hydrophobic Polymers

Table 2 describes lipid vesicles containing single and dual chain lipids.

TABLE 2
Polymeric lipid vesicles with single and dual chained lipids

| | | | | | |
|---|---|---|---|---|---|
| Component | 1 | 2 | 3 | 4 | Ranges % |

Lipid Phase

| | | | | | |
|---|---|---|---|---|---|
| Glyceryl | 20 | 0 | 0 | 45 | 0-90 |
| distearate | | | | | |
| Glyceryl Dilaurate | 15 | 20 | 5 | 0 | 0-90 |
| Cholesterol | 5 | 0 | 10 | 0 | 0-60 |
| Bisabolol | 0 | 5 | 0 | 15 | 0-60 |
| Polyoxyethylene- | 0 | 2.0 | 20 | 0 | 0-80 |
| 10-stearyl ether | | | | | |
| Polyoxyethylene- | 10 | 2.0 | 0 | 0 | 0-40 |
| 10-octyl ether | | | | | |
| PEG-8-dilaurate | 2 | 0 | 20 | 10 | 0-60 |
| DSHM* | 1 | 0 | 1 | 0 | 0-60 |
| Quaternium 16** | 0 | 10 | 0 | 5 | 0-40 |
| Polyoxyethylene- | 0 | 5 | 4 | 1 | 0-40 |
| 100-stearyl ether | | | | | |
| Methylcellulose | 23.5 | 6 | 40 | 0 | 0-40 |
| Phenyl | 23.5 | 50 | 0 | 24 | 0-80 |
| Trimethicone | | | | | |
| Sub Total | 100 | 100 | 100 | 100 | 100 |

Aqueous Phase

| | | | | | |
|---|---|---|---|---|---|
| Di water | 69.6 | 69.6 | 69.6 | 84.6 | 10-99.99 |
| Methyl Paraben | 0.2 | 0.2 | 0.2 | 0.2 | 0-2 |
| Propyl Paraben | 0.2 | 0.2 | 0.2 | 0.2 | 0-2 |
| PVP K-30 | 30 | 0 | 0 | 5 | 0-80 |
| Hydroxyethyl- | 0 | 30 | 10 | 5 | 0-60 |
| cellulose | | | | | |
| Polyquaternium 10 | 0 | 0 | 20 | 5 | |
| Sub Total | 100 | 100 | 100 | 100 | 100 |

*Dual Chain Cationic lipid: Di (soyoylethyl) hydroxyethylmonium methosulfate
**Single Chain Cationic Lipid: triethyl hydrogenated tallow ammonium chloride

Each of the above formulations is made by mixing appropriate amounts of the components of the lipid phase in a beaker a 65° C. until the lipids melted. The resulting melt is then drawn into a syringe, which is preheated in a water-bath to 65° C. In a second beaker the aqueous phase is combined and mixed until uniform. A second syringe the aqueous phase is preheated in a water-bath to 60° C. The two syringes are then connected via a 3-way metal stopcock. The ratio of aqueous phase to lipid phase is about 80:20 or 8 ml of aqueous phase to 2 ml of lipid phase. After injecting the aqueous phase components into the lipid phase syringe, the resulting mixture is rapidly mixed back and forth between the two syringes several times until the contents cooled to about 25-30° C.

### EXAMPLE 3

Preparation of Liposomal-Polymeric Conditioner Compositions

Liposomal Polymer conditioner compositions comprised of the following components as set forth in Tables 3, 4 and 5 were prepared as follows:

TABLE 3
Liposomal PVP Conditioner for increasing volume of hair

| | | Range | Trade- | |
|---|---|---|---|---|
| Component | Wt % | (% W/W) | name | Supplier |
| Di Water | 93.4 | 20-99.9 | — | — |
| Polyquaternium-37, | 1.50 | 0.05-10 | Salcare | Ciba |
| Propylene Glycol | | | SC96 | |
| Dicaprylate/ | | | | |
| Dicaprate, PPG-1 | | | | |
| Trideceth-6 | | | | |
| PVP Liposomes | 2.00 | 0.01-95 | — | Example 1 |
| | | | | Formula 1 |
| Glycerin | 1.00 | 0.01-60 | Glycerine | Condor |
| | | | | Corp |
| Phenoxyethanol | 1.00 | 0.01-5 | Phenonip | Nipa |
| Methylparaben, | | | | Hardwick |
| Ethylparaben, | | | | Inc |
| Propylparaben, | | | | |
| Butylparaben, | | | | |
| Isobutylparaben | | | | |
| Polyquaternium-10 | 0.1 | 0.05-10 | Ucare | Amerchol |
| | | | Polymer | Corp |
| | | | JR-30 | |
| Panthenol | 0.5 | 0.01-2 | Pantheno | Hoffman La |
| | | | 1-50 | Roche |
| PVP | 0.2 | 0.01-5 | PVP K-30 | BASF Corp |
| Fragrance | 0.3 | 0.05-1 | Fragrance | Givaudan |
| | | | | Roure |

The Deionized water was added in a suitable container. The Ucare Polymer JR-30 was dispersed in the water with moderate agitation for approximately 10 minutes. When the solution was homogeneous, the Salcare SC96 was then added and mixed until the mixture was free of lumps, usually 15 minutes. The mixture was then heated to 60° C. The PVP liposomes were then added and mixed at 500 rpm for 10 minutes. The additional PVP K-30, panthenol, phenonip, and glycerin were then added to the mixture. The composition was then cooled to 35° C. and the fragrance was then added by mixing at 100 rpm for 5 minutes. The resulting composition was then cooled to room temperature.

TABLE 4
Liposomal PVP and liposomal Phenyl Trimethicone
conditioner for increasing shine of hair

| | Range | | |
|---|---|---|---|
| Component | (% W/W) | Tradename | Supplier |
| DI Water | 40-99.9 | — | — |
| Phenyl Trimethicone | 0.01-10 | DC 556 | Dow Corning |
| Phenyl Trimethicone | 0.001-95 | — | Example 1 |
| Liposomes | | | Formula 2 |
| Glycerin | 0-60 | Glycerine | Condor Corp |
| Polyquaternium-37 | 0.01-10 | Salcare | Ciba |
| Propylene Glycol | | SC96 | |
| Dicaprylate/Dicaprate | | | |
| PPG-1 Trideceth-6 | | | |
| Panthenol | 0.01-5 | Panthenol | Hoffman La |
| | | -50 | Roche |
| PVP Liposomes | 0.001-95 | — | Example 1 |
| (table 1) | | | Formula 1 |
| Phenoxyethanol | 0.01-5 | Phenonip | Nipa Hardwick |
| Methylparaben | | | |
| Ethylparaben | | | |
| Propylparaben | | | |
| Butylparaben | | | |
| Isobutylparaben | | | |
| Triethanolamine | 0.001-2 | TEA 99% | Van Waters & |
| Fragrance | 0.001-2 | Fragrance | Givaudan |
| | | | Roure |

The Deionized water was added in a suitable container. The Salcare SC96 was then added and mixed for approximately 10 minutes or until the mixture was free of lumps. The mixture was then heated to 60° C. The phenyl trimethicone liposomes and PVP liposomes were then added and mixed at 500 rpm for 10 minutes. The phenyl trimethicone, glycerin, panthenol, triethanolamine and phenonip were then added and mixed while being cooled to 35° C. At 35° C. the fragrance was then added and mixed for 5 minutes. The composition was then cooled down to room temperature.

TABLE 5
Liposomal PVP conditioner for deep conditioning of hair

| | Range | Trade- | |
|---|---|---|---|
| Component | (% W/W) | name | Supplier |
| Water | 40-99.9 | — | — |
| Hydroxyethyl- | 0.01-5 | Natrosol | Aqualon |
| cellulose | | HHR | |
| Methylparaben | 0.01-2 | Methylpar | Ueno |
| | | aben | |
| Propylparaben | 0.01-2 | Propylpar | Ueno |
| | | aben | |
| Behentrimonium | 0.01-6 | Incroquat | Croda |
| Methosulfate (and) | | Behenyl | |
| Cetearyl Alcohol | | TMS | |
| Cetyl Alcohol | 0.05-10 | Cetal | Amerchol |
| Polyquaternium 37 | 0.05-10 | Salcare | Ciba |
| Propylene Glycol | | SC 96 | |
| Dicaprylate/Dicaprate | | | |
| PPG-1 Trideceth-6 | | | |
| Dimethicone | 0.01-40 | Dow | Dow Corning |
| | | Corning | |
| | | 200 Fluid | |
| Hydrolyzed Wheat | 0.01-10 | Crodasone | Croda |
| Protein PG-Propyl | | W | |
| Silanetriol | | | |
| Hydrolyzed Wheat | 0.01-10 | Cropeptide | Croda |
| Protein (and) | | W | |
| Hydrolyzed Wheat | | | |
| Starch | | | |
| DL Panthenol | 0.01-10 | DL | Hoffman La |
| | | Panthenol | Roche |
| | | 50% | |
| Phenoxyethanol | 0.001-2 | Phenoxyet | Nipa/Dow |
| | | hanol | |
| Fragrance | 0.001-2 | Fragrance | Givaudan |
| | | | Roure |
| Water | 1-99.5 | — | — |
| Liposome PVP | 0.001- | — | Example 1 |
| | 99.5 | | Formula 1 |

Deionized water was charged in a suitable beaker. The hydroxyethylcellulose was dispersed by mixing at 500 rpm for 10 minutes. The mixture was then heated to 70° C.

Further mixing was required until the hydroxyethyl-cellulose was fully hydrated and the mixture turned clear, approximately 10 minutes. Then methylparaben, propylparaben, behentrimonium methosulfate (and) cetearyl alcohol, and cetyl alcohol was added at 70° C. and mixed for 5 minutes. The resulting mixture was then cooled to 60° C. and the Salcare SC 96 was then added. The mixture was mixed at 500 rpm for 5 minutes or until the mixture turned homogeneous. Dimethicone was then added, mixed and cooled to 40° C. At 40° C., the Crodasone W, Cropeptide W, DL Panthenol 50% and phenoxyethanol mixed for 10 minutes. The remaining Di water the PVP liposomes were then added and mixed for 5 minutes. The composition was then cooled to 25° C.

### EXAMPLE 4

Preparation of Liposomal-Polymer Shampoo

Liposomal Polymer shampoo compositions comprised of the following components as set forth in Table 6 were prepared as follows:

TABLE 6
Liposomal PVP shampoo

| | Range | | |
|---|---|---|---|
| Component | (% W/W) | Tradename | Supplier |
| Water | 10-99.5 | — | — |
| Polyquaternium | 0.01-5 | UCARE Polymer | Amerchol |
| 10 | | LK 400 | |
| Methylparaben | 0.01-5 | Methylparaben | Ueno |
| Propylparaben | 0.001-5 | Propylparaben | Ueno |
| Cocamide MEA | 0.01-20 | Monamid CMA | Lipscomb |
| Ammonium Lauryl | 0.01-50 | Sulfochem ALS | Chemron |
| Sulfate | | | |
| Ammonium | 0.01-50 | Standapol EA- | Cognis |
| Laureth Sulfate | | 2 | |
| Cocamidopropyl | 0.01-20 | Chembetaine | Chemron |
| Betaine | | CGF | |
| Glycol | 0.01-10 | Lexemol EGDS | Inolex |
| Distearate | | | |
| Cetyl Alcohol | 0.01-10 | Cetal | Amerchol |
| Dimethicone | 0.01-20 | Silsoft A-843 | Witco |
| Bisamino | | | |
| Hydroxypropyl | | | |
| Copolyol | | | |
| DL Panthenol | 0.01-2 | DL Panthenol | Hoffman La |
| | | 50% | Roche |
| Tetrasodium | 0.01-2 | Hampene 220 | Hampshire |
| EDTA | | | |
| Phenoxyethanol | 0.01-5 | Phenoxyethanol | Nipa/Dow |
| Water | 0-50 | — | — |
| Liposome PVP | | — | Example 1, |
| | | | Table 1, F#1 |
| BHT | 0.001-1 | Naugard BHT | Uniroyal |
| Fragrance | 0.01-1 | Fragrance | Givaudan |
| Citric Acid | 0.01-2 | Citric Acid | H&R |

A vessel was charged with Deionized water (first charge). The Ucare polymer LK 400 was then dispersed in the water by mixing at 500 rpm for 10 minutes or until the mixture turned clear. The mixture was then heated to 75° C. and the methylparaben, propylparaben and the cocamide MEA were added and mixed at 500 rpm for 5 minutes or until those components were dissolved. Ammonium lauryl sulfate, ammonium laureth sulfate, and the cocamidopropyl betaine were then added and mixed after each addition for 5 minutes. The mixture was then cooled to 60° C. and the glycol distearate and cetyl alcohol was then added and mixed until those components dissolved, usually 10 minutes at 500 rpm. The mixture was then cooled to 50° C. and the following items were added: Silsoft A-843, DL panthenol, tetrasodium EDTA, and phenoxyethanol, and the combination was mixed well. The rest of the water was then added along with the PVP liposomes. The mixture was then cooled to 40° C. and the BHT and fragrance was added and mixed for 5 minutes. Finally, the citric acid was added and mixed at 500 rpm for 5 minutes.

### EXAMPLE 5

Freeze Fracture Microscopy

The compositions of Examples 1, 3 and 4 were examined using a freeze-fracture transmission electron microscope (FF-TEM), freshly made and after storage at elevated temperatures. FF-TEM samples of each formulation were prepared in accordance with techniques described in chapter 5 of “Low Temperature Microscopy and Analysis” by Patrick Echlin (1992), which is incorporated by reference herein. The samples were fractured at low temperature and etched at −150° C. for purposes of removing a surface layer of water.

The photomicrographs of compositions of Example 1 showed the presence of large bilayered structures ranging in size from 100 nm to 800 nm, which were stable upon product storage at 50° C. for 4 weeks. The photomicrograph of the compositions of Example 3, which contained PVP liposomes and/or phenyl trimethicone liposomes in a conditioning base showed the presence of intact vesicles with many bilayers. The photomicrograph of the compositions of Example 4, which contained PVP liposomes and/or phenyl trimethicone liposomes in a shampoo base showed the presence of intact vesicles with many bilayers, even in the presence of detergents.

Thus, the freeze-fracture photomicrographs showed that the lipid vesicles remained intact as formulated, and after accelerated aging storage.

### EXAMPLE 6

Consumer Home Use Study

Formulas 1 and 2 of Example 3 containing PVP liposomes were used in the following consumer home study. Each composition was evaluated monadically by 100 respondents who graded the compositions on a number of attributes. The panelists were asked to apply the compositions once and then wash their hair once a day for seven days. Table 7 shows the results from the test (percentage of respondents that completely or somewhat agree). The difference in the PVP liposomes is the levels of the dual chained cationic lipid in the liposomal bilayers of the composition; (Formula 1 has a higher level of cationic lipid versus formula 2).

TABLE 7

| Attributes | Formula 1 | Formula 2 |
|---|---|---|
| Hair has noticeable volume | 67% | 56% |
| after use | | |
| Gives Hair volume for 7 days | 60% | 47% |
| Leaves hair shinier | 68% | 58% |
| Leaves hair more touchable | 69% | 58% |
| Leaves hair more manageable | 67% | 62% |
| Leaves hair healthier looking | 69% | 59% |
| Does not leave an unpleasant | 75% | 71% |
| coating on hair | | |
| Is an effective volumizing | 71% | 65% |
| product | | |

It is evident from this data above that the two formulas can delivery consumer conditioning benefits that can last for several days. This was very unexpected since most products specific for increasing volume or adding body to the hair usually disappear after one shampooing. A large percentage of the panelist responded to experience a volumizing effect for 7 days, indicated that the mechanism of action may be more complex that just a simple surface coating phenomena.

### EXAMPLE 7

Preparation of Fluorescent Labeled Polyvinylpyrrolidone (PVP)

Fluorescein with a hydrazine derivative was reacted with polyvinylpyrrolidone K-30 (average molecular weight 42,000) (PVP) via a Schiff Base Formation reaction as described on Chapter 2, The Chemistry of Reactive Groups in Bioconjugate Techniques, Academy Press, New York, ed. Greg Hermanson, 1996 (page 186). The hydrazine will react with the carbonyl group of the PVP to form a stable conjugate. A 50:1 molar ratio of fluorescent hydrazine derivative to PVP was mixed in the in a 0.1M sodium Dorate, pH 9.5 buffer. The reaction progressed for 8 hours. The mixture was then ultracentifuged at 25,000 rpm for 2 hours. The free hydrazine fluorescent reagent phase separated from the PVP polymer and the free reagent was removed. The labeled PVP was ultracentifuged again for 4 hours at 25,000 rpm. Again the phase separated free reagent was removed, and the labeled PVP was ultracentifuged one more time at 25, 000 rpm for 8 hours.

### EXAMPLE 8

Fluorescence Microscopy of Hair Tresses

The following formulas of Table 8 were used in this example.

TABLE 8
Formulas in Hair Tress studies

| | Description of | | Conditioning |
|---|---|---|---|
| Formula # | Formula | Liposomes | Base |
| Formula 1 | Liposomal PVP | Example 1 | Example 3 |
| | with low amount | Table 1 | Table 3 |
| | cationic lipid in | F#2 | |
| | lipid bilayers | | |
| Formula 2 | Liposomal PVP | Example 1 | Example 3 |
| | with high amount | Table 1 | Table 3 |
| | cationic lipid in | F#1 | |
| | lipid bilayers | | |
| Formula 3 | No liposomes, | — | Example 3 |
| | free PVP | | Table 3 |

Each formula was prepared using the fluorescent-labeled PVP of Example 7. The fluorescent labeled PVP was self-quenching when encapsulated inside the liposomal systems. Fluorescent-PVP not encapsulated in the liposomal system revealed the fluorescent signal and was not quenched.

Briefly, each formula of Table 8 was applied separately to virgin hair tress sections (approximately 6-7 g total weight) for 5 minutes. The hair tresses were then rinsed for 10 minutes with 37° C. distilled water and allowed to dry overnight. The hair tresses were then washed with 0.1 g of Neutrogena Clean shampoo 7 times and blown dry with a hair drier between washings. Hairs were collected randomly from the tresses after each washing cycle. The hairs were then examined using a fluorescent Microscope (Leitz GmbH, Germany) with a color chilled 3CCD camera (Hammamatsa, Japan). The microscope was equipped with a filter for detecting fluorescein. The images were captured using Image Pro software (Media Cybernetics, Md.).

Hairs obtained from the hair tresses were also frozen in Tissue Tek OCT compound (Sakura Fineteck, Calif.) and sectioned via a cryostat (Microm GmbH, Germany) to 5 μm thickness. These sections were also examined using a fluorescent microscope. The cross-sectional hair morphology was observed using a fluorescent microscope equipped with a filter specific for fluorescein.

Formula 1 revealed that after one washing, very little fluorescence was seen over the surface of the hair fibers and inside the hair fibers. However, after three washing a tremendous amount of fluorescent was observed on the hair surface and inside the hair shaft. After five washings, the fluorescent signal inside the hair shaft was diminished as compared to 3 washes. However, the signal was retained on the outside of the hairs perhaps because of migration of fluorescent PVP from the inside of the hair fibers to the outer surface of the hair fibers. After seven washings, the amount of fluorescence was further diminishing, but the signal was still present in the inside and on the outside of the hair as compared to placebo.

Formula 2 results were similar to that of formula 1, however, the amount of fluorescence on the outside of the hair fibers was of greater intensity as compared to formula 1. This possibly indicates a greater amount of binding of the liposomes on the outside of the hair fibers due to the greater amount of cationic charges in the liposomal composition interacting with the negative charges of the keratin fibers in the hair, as well as fluoresent PVP delivered inside the hair fibers.

The free PVP composition of formula 3 showed fluorescence on the outside of the hair and much less inside the hair. After three washings, most of the fluorescent signal was gone indicating that PVP alone, not encapsulated in liposomes, is not effective in retaining PVP on or in the hair fibers over several washings. By five washings, no fluorescent signal was present inside or outside the hair fibers, indicating no retention of PVP by the hair fibers.

This data indicates that liposomes are effectively delivering PVP, a polymeric hair volumizing agent, onto and into the hair fibers, possibly indicating that the internalized PVP is available as a reservoir and slowly releases to the surface of the hair as the PVP is depleted from the surface by subsequent hair washing. The effect is demonstrable by the fluorescent microscopy technique over 3 to 5 subsequent shampooings.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A composition for application to the hair or skin wherein said composition comprises a conditioning polymer encapsulated in a lipid vesicle.

2. A composition of claim 1, wherein said polymer is a polyvinylpyrrolidone.

3. A composition of claim 1, wherein said polymer is a polyoxyethylene ether.

4. A composition of claim 1, wherein said polymer is a polymer of hyaluronic acid.

5. A composition of claim 1, wherein said lipid vesicle comprises a dual chain lipid and a hydrophilic liquid.

6. A composition of claim 2, wherein said lipid vesicle comprises a dual chain lipid and a hydrophilic liquid.

7. A composition of claim 3, wherein said lipid vesicle comprises a dual chain lipid and a hydrophilic liquid.

8. A composition of claim 4, wherein said lipid vesicle comprises a dual chain lipid and a hydrophilic liquid.

9. A composition of claim 5, wherein said lipid vesicle comprises:
(a) a dual chain lipid which is a glyceryl diester, an alkoxylated amine, or a mixture thereof;
(b) a single chain lipid which is a glyceryl monoester, a polyoxyethylene fatty ether, or a mixture thereof;
(c) a sterol which is cholesterol; and
(d) a hydrophilic liquid which is water.

10. A composition of claim 6, wherein said lipid vesicle comprises:
(a) a dual chain lipid which is a glyceryl diester, an alkoxylated amine, or a mixture thereof;
(b) a single chain lipid which is a glyceryl monoester, a polyoxyethylene fatty ether, or a mixture thereof;
(c) a sterol which is cholesterol; and
(d) a hydrophilic liquid which is water.

11. A composition of claim 7, wherein said lipid vesicle comprises:
(a) a dual chain lipid which is a glyceryl diester, an alkoxylated amine, or a mixture thereof;
(b) a single chain lipid which is a glyceryl monoester, a polyoxyethylene fatty ether, or a mixture thereof;
(c) a sterol which is cholesterol; and
(d) a hydrophilic liquid which is water.

12. A composition of claim 8, wherein said lipid vesicle comprises:
(a) a dual chain lipid which is a glyceryl diester, an alkoxylated amine, or a mixture thereof;
(b) a single chain lipid which is a glyceryl monoester, a polyoxyethylene fatty ether, or a mixture thereof;
(c) a sterol which is cholesterol; and
(d) a hydrophilic liquid which is water.

13. A composition of claim 1, wherein said composition further comprises a detergent.

14. A method of conditioning, strengthening, or moisturizing the hair, said method comprising topically applying a composition comprises a conditioning polymer encapsulated in a lipid vesicle.

15. A method of claim 14, wherein said polymer is a polyvinylpyrrolidone.

16. A method of claim 14, wherein said polymer is a polyoxyethylene ether.

17. A method of claim 14, wherein said lipid vesicle comprises a dual chain lipid and a hydrophilic liquid.

18. A method of claim 15, wherein said lipid vesicle comprises a dual chain lipid and a hydrophilic liquid.

19. A method of claim 16, wherein said lipid vesicle comprises a dual chain lipid and a hydrophilic liquid.

20. A method of claim 17, wherein said lipid vesicle comprises:
(a) a dual chain lipid which is a glyceryl diester, an alkoxylated amine, or a mixture thereof;
(b) a single chain lipid which is a glyceryl monoester, a polyoxyethylene fatty ether, or a mixture thereof;
(c) a sterol which is cholesterol; and
(d) a hydrophilic liquid which is water.

21. A method of claim 18, wherein said lipid vesicle comprises:
(a) a dual chain lipid which is a glyceryl diester, an alkoxylated amine, or a mixture thereof;
(b) a single chain lipid which is a glyceryl monoester, a polyoxyethylene fatty ether, or a mixture thereof;
(c) a sterol which is cholesterol; and
(d) a hydrophilic liquid which is water.

22. A method of claim 19, wherein said lipid vesicle comprises;
(a) a dual chain lipid which is a glyceryl diester, an alkoxylated amine, or a mixture thereof;
(b) a single chain lipid which is a glyceryl monoester, a polyoxyethylene fatty ether, or a mixture thereof;
(c) a sterol which is cholesterol; and
(d) a hydrophilic liquid which is water.

23. A method of claim 14, wherein said composition further comprises a detergent.
